(19) United States
(12) Patent Application Publication
(10) Pub. No.: US 2003/0045587 A1
(43) Pub. Date: Mar. 6, 2003

(54) Solvent system

(76) Inventor: David Anderson, Colonial Heights, VA (US)

Correspondence Address: Whitham, Curtis & Christofferson, PC, 11491 Sunset Hills Road, Suite 340, Reston, VA, 20190, US

(21) Appl. No.: 10/170,236

(22) Filed: Jun. 13, 2002

Related U.S. Application Data
(60) Provisional application No. 60/300,482, filed on Jun. 23, 2001.

(51) Int. Cl. A61K 47/00

(52) U.S. Cl. 514/772; 514/777

(57) ABSTRACT

A solvent system for the solution in a polar solvent of a poorly soluble compound having pharmacological activity is disclosed that comprises a first polar organic material comprising a sugar having at least two hydroxyl groups or an alcohol of two or three carbons having at least two hydroxyl groups or both and a second polar organic material comprising an amide group or an ammonium group or both., such as, one or more of acetamide, ethylammonium nitrate, N-methylacetamide and dimethylacetamide, preferably an amide.

### FIELD OF THE INVENTION

The present invention relates to solvent systems for poorly soluble compounds having pharmacological activity, including both lipid-based systems and systems not based on lipids.

### BACKGROUND OF THE INVENTION

A number of compounds having pharmacological activity have been found to be difficult to dissolve into aqueous solution, despite containing several polar groups in their molecular structure, in fact, often on the order of 4-12 polar groups per molecule. For example, sodium 1-[[[5-(4-Nitrophenyl)-2-furanyl]-methylene]amino]-2,4-imidazolidinedione hemiheptahydrate (sodium dantrolene) is poorly soluble in water. A single 300 mg dose requires approximately 1 liter of water for complete dissolution. Further, the presence of a number of polar groups on the molecule is indicative of low solubility in apolar liquids, and even surfactant-rich or lipid-rich mixtures, low enough that solubilization of the compound in typical liposomes, micellar solutions, emulsions and the like is impractical in terms of delivery of therapeutically effective dosage.

Also, it is not always sufficient to merely solubilize a given drug, even if it is in a non-toxic vehicle; the vehicle must lend itself to whatever transformation—e.g., encapsulation, enteric coating, freeze- or spray-drying—is required to arrive at the correct delivery format. For example, for pharmaceutical actives where the most desirable format is the pill form for oral delivery, still the most common drug format by far, most liquid solvents and even surfactants, unless encapsulated, will often be incompatible with the simplest tablet manufacturing procedures, since these procedures were generally developed with solids and powders in mind. Yet the application of these procedures to poorly-soluble drugs without the use of liquids or surfactants often yields a pill that achieves only a very limited bioavailability when administered. It should also be pointed out that while acidic (e.g., hydrochloride) or basic (e.g., sodium) salt forms of low-solubility drugs can often be soluble, such salts can precipitate in the body when they encounter pH conditions that deprotonate the acidic salt or protonate the basic salt. In the case of delivery of drugs by injection, rapid precipitation of drug on contact with water for reconstitution or with blood can lead to fatal emboli, underscoring the need for superior solubilization and encapsulation systems.

Nanostructured liquid crystalline phases, and particularly those of the reversed type—namely reversed cubic and reversed hexagonal phases—can be of very low solubility in water, meaning that they maintain their integrity as vehicles upon entry into the body thus avoiding drug precipitation, and show a great deal of promise in fields such as controlled-release drug delivery. In work motivated by the amphiphilic nature and porous nanostructures or these reversed liquid crystalline materials, which could lead to very advantageous interactions with biomembranes—much more intimate than in the case of liposomes—and by the high viscosities of these phases which can be an important aid in processing, a number of techniques have been developed for encapsulating such phases.

However, a limitation in previous attempts to use reversed liquid crystalline phases in the solubilization of pharmaceutical actives has come about because of the tacit, and frequently incorrect, assumption that a drug of low solubility in water should be hydrophobic and should thus be soluble in lipid, or in a binary (or pseudo-binary) lipid-water system. But the application of simple surfactant-water or lipid-water binary systems to the solubilization of many difficultly-soluble drugs has met with very limited success.

### SUMMARY OF THE INVENTION

The present invention provides a composition of matter comprised of a poorly soluble compound (for example, a compound requiring more than about 100 ml of water to effect solubilization of a therapeutic amount of the compound), and a solvent system capable of effecting solubilization of the compound. Suitable solvent systems take advantage of, or are based largely on, lipids or surfactants and their ability to form self-association structures, nanostructured liquid and liquid crystalline phases that are uniquely well suited for pharmaceutical formulations that promote absorption, exhibit biocompatibility and even biofunctionality, and permit relatively simple and inexpensive processing despite sophisticated functionality. The solvent systems comprise mixtures of polar, low-MW amide liquids such as dimethylacetamide with low-MW, hydroxyl-rich compounds such as glycerol and sugars which exhibit synergy in the formulation of difficultly-soluble pharmaceutical actives, not only in the ability to solubilize difficultly soluble actives with low-toxicity, water-miscible solvent mixtures, but also in the compatibility of such mixtures with surfactant and lipid self-association structures and liquid crystals. In particular, the invention provides a non-aqueous solvent system comprising: a first polar organic material including a sugar having at least two hydroxyl groups or an alcohol of two or three carbons having at least two hydroxyl groups, or both; and a second polar organic material comprising an amide group or an ammonium group, or both. The composition may further include a lipid material or a surfactant material, or both.

The solvent system may be non-aqueous; the first polar organic material may have a molecular weight of less than about 150.

The first polar organic material may have a ratio of hydroxyl groups to carbon atoms of from about 1:2 to about 1:1. Alternatively, the first polar organic material may have a ratio of hydroxyl groups to carbon atoms of from about 3:5 to about 1:1, or a ratio of hydroxyl groups to carbon atoms of from about 2:3 to about 1:1. The first polar organic material may comprise a sugar, or a dihydric or trihydric alcohol of two or three carbons, which may be a glycerol or a glycol.

The second polar organic material may include an hydroxyl-free polar organic material comprising an amide group or an ammonium group, or both. In preferred embodiments, the second polar organic material comprises an amide, which may comprise an acetamide or N,N-dimethylacetamide.

The poorly soluble compound may have one or more polar groups in its molecular structure, including: a hydantoin-group; an amide group and one polar atom, N or S, separated by a single carbon and a nitrophenyl- or nitrofuranyl-group; an amide group and one polar atom, N or S, separated by a single carbon; two nitrogen atoms separated from each other by a single carbon; an amide group as part of a 5-member ring; both a hydantoin- and a nitrophenyl- or nitrofuranyl-group; a nitrophenyl- or nitrofuranyl-group and a —C═N—N—C═O— group; a nitrophenyl- or nitrofuranyl-group; a terminal nitrofuranyl-group, and two nitrogens separated from each other by a single carbon and; a plurality of nitrophenyl- or nitrofuranyl-groups.

With respect to the composition of matter comprised of a poorly soluble compound and a solvent system capable of effecting solubilization of the compound, the ratio of the first polar organic material to the second liquid polar organic material, as a weight ratio, may be in a range from about 1:2 to about 10:1, or in a range from about 1:1 to about 5:1, or in a range from about 3:2 to about 4:1. Further, one or the other or both of the first and second polar organic materials may be a liquid.

In a preferred embodiment, the present invention provides a solvent system for the solution in a polar solvent of a poorly soluble compound having pharmacological activity. The solvent system includes: a dihydric or trihydric alcohol of two or three carbons; and, one or more of acetamide, ethylammonium nitrate, N-methylacetamide and dimethylacetamide. The solvent system may contain a surfactant, and the method of administration may be oral.

In yet another aspect of the invention, a solvent system for the solution of compounds containing hydantoin groups, (such as dantrolene and dilantin) in a polar solvent is provided. The solvent system includes: a dihydric or trihydric alcohol of two or three carbons; and, a polar organic material comprising an amide group or an ammonium group, or both. In one embodiment, the solvent system comprises 1,2,3-propanetriol and N,N-dimethylacetamide.

The present invention also provides solvent system for the solution of dantrolene or dilantin in a polar solvent. The system includes: a dihydric or trihydric alcohol of two or three carbons; and a polar organic material comprising an amide group or an ammonium group, or both. The solvent system may comprise 1,2,3-propanetriol and N,N-dimethylacetamide.

The present invention also provides a method for solubilizing a poorly soluble compound (i.e. requiring more than about 100 ml of water to effect solubilization of a therapeutic amount of the compound) having pharmacological activity. The method includes the step of combining the poorly soluble compound with a solvent system comprising a first polar organic material comprising a sugar having at least two hydroxyl groups or an alcohol of two or three carbons having at least two hydroxyl groups or both; and, a second polar organic material comprising an amide group or an ammonium group or both. The solvent system is present in an amount sufficient to solubilize the poorly soluble compound.

The present invention also encompasses a method of providing to a patient in need thereof a poorly soluble compound having pharmacological activity. This is accomplished by administering to the patient a pharmaceutical composition comprising: the poorly soluble compound; and, a solvent system comprising a first polar organic material comprising a sugar having at least two hydroxyl groups or an alcohol of two or three carbons having at least two hydroxyl groups, or both; and a second polar organic material comprising an amide group or an ammonium group or both.

A pharmaceutical composition comprising dantrolene or salts thereof, and a solvent system is also provided. The solvent system includes a first polar organic material comprising a sugar having at least two hydroxyl groups or an alcohol of two or three carbons having at least two hydroxyl groups, or both; and, a second polar organic material comprising an amide group or an ammonium group, or both. The dantrolene is dissolved in the solvent system and is present in a range of from about 0.3% to about 25%, whereas the solvent system is present in a range of from about 1 to about 99%. In one embodiment, the first polar organic material is glycerol, a sugar or a plurality of sugars. In another embodiment, the second polar organic material is acetamide, N-methylacetamide, or N,N-dimethylacetamide.

The present invention also provides a method of providing dantrolene to a patient in need thereof. The method comprises the step of administering to said patient a pharmaceutical composition comprising: dantrolene or salts thereof; and a solvent system comprising a first polar organic material comprising a sugar having at least two hydroxyl groups or an alcohol of two or three carbons having at least two hydroxyl groups, or both; and a second polar organic material comprising an amide group or an ammonium group, or both. The dantrolene is dissolved in the solvent system.

### DETAILED DESCRIPTION OF THE PREFERRED EMBODIMENTS OF THE INVENTION

The present invention is based on the discovery of solvent systems that are capable of effecting the solubilization of poorly soluble compounds. The solvent systems are composed of substances that exhibit relatively low or no toxicity, and possess the ability to solubilize useful amounts of poorly soluble compounds, particularly compounds with pharmaceutical applications. A solvent system in accordance with the present invention comprises

a. a first polar organic material comprising a sugar having at least two hydroxyl groups or an alcohol of two or three carbons having at least two hydroxyl groups, or both and

b. a second polar organic material comprising an amide group or an ammonium group or both; preferably, said polar organic material comprising an hydroxyl-free polar organic material comprising an amide group or an ammonium group or both, and desirably solubilizes poorly soluble compounds having polar groups. More preferably, the polar organic material comprises a liquid amide.

The solvent system of the present invention may further comprise a lipid material or a surfactant material or both.

Further, a solvent system in accordance with the present invention desirably solubilizes poorly soluble compounds having polar groups, such as,

1. a hydantoin-group;

2. an amide group and one polar atom, N or S, separated by a single carbon and a nitrophenyl- or nitrofuranyl-group;

3. an amide group and one polar atom, N or S, separated by a single carbon;

4. two nitrogen atoms separated from each other by a single carbon;

5. an amide group as part of a 5-member ring;

6. both a hydantoin- and a nitrophenyl- or nitrofuranyl-group;

7. a nitrophenyl- or nitrofuranyl-group and a —C═N—N—C═O— group;

8. a nitrophenyl- or nitrofuranyl-group;

9. a terminal nitrofuranyl-group, and two nitrogens separated from each other by a single carbon and;

10. a plurality of nitrophenyl- or nitrofuranyl-groups.

In a preferred embodiment a solvent system for the solution of dantrolene or dilantin in a polar solvent comprises

a. 1,2,3-propanetriol (glycerol) and

b. N,N-dimethylacetamide.

For the case of such a compound that is to be delivered by injection, such a compound is deemed to be poorly soluble in water if more than about 100 ml. of water are required to solubilize a therapeutic amount of the compound. By “water” is meant either unbuffered water or water buffered at or near physiological pH, i.e., about 7.4.

An important aspect of this invention is that it focuses on solvent systems of low toxicity, in particular of low enough toxicity that many embodiments are composed entirely of materials that are approved by the FDA for use in injectable products. In 1996, the Division of Drug Information Resources of the Food and Drug Administration published the Inactive Ingredient Guide, in which it tabulated those mateirals that were pharmaceutically-acceptable for formulations intended for various routes of administration, including a number that were approved for injectable routes (intravenous, subcutaneous, intramuscular). In addition to these, several other excipient materials have appeared in products approved for marketing in the United States. Thus, as a particularly important example of this, dimethylacetamide is approved for use in an injectable product (Vumon^{R}) that is on the market at the time of this writing, and at levels of several grams per injection. Likewise, many sugars and hydroxyl-rich compounds like glycerol are approved for use in injectable formulations at high levels (for example, glycerol comprises 70% w/v of the formulation Multitest CMI^{R}) . In contrast with this, solubilization of difficult drugs in non-approved solvents like dimethylsulfoxide (DMSO), commonly used in research, is of little or no utility in the adminstration of therapeutic agents to mammals or especially to man. Thus, we define pharmaceutically-acceptable materials to be those that appear either on the 1996 Inactive Ingredient Guide, or in the Physician's Desk Reference of year 2001 (that is, current as of the time of this writing), indicating that they appear in a currently-marketed formulation as of this fixed moment in time, or in both publications. Similarly, we define “pharmaceutically-acceptable for injection” to be those materials that appear either on the 1996 Inactive Ingredient Guide as being approved for injectable formulations, or in one or more injectable formulations in the Physician's Desk Reference of year 2001, or both.

The following Table 1 lists a number of compounds having pharmacological activity with relatively low solubility in both water and lipid and that are problematic when formulated as salts (e.g. requiring strongly acidic or alkaline solutions, encountering precipitation or low absorption as a result of pH requirements, causing GI upset, etc.) and a tabulation of the polar groups contained on each molecule:

TABLE 1

| | Therapeutic | | | | | | | | | |
|---|---|---|---|---|---|---|---|---|---|---|
| Compound | category | Amino | Hydroxyl | Carboxyl | Amide | Carbonyl | Phenolic | Cation | Other | Total |
| Enalapril | ACE Inhibitor | 1 | | 1 | 1 | 1 | | | | 4 |
| Albuterol | β-Andregenic | 1 | 2 | | | | 1 | | | 4 |
| | agonist | | | | | | | | | |
| Sulfinalol | β-Andregenic | 1 | 1 | | | | 1 | | 2 | 5 |
| | blocker | | | | | | | | | |
| Nandrolone | Anabolic | | 1 | | | 1 | | | | 2 |
| Morphine | Analgesic | 1 | 1 | | | | 1 | | 1 | 4 |
| | (narcotic) | | | | | | | | | |
| Aspirin | Analgesic | | | 1 | | 1 | | | | 2 |
| | (non-narcotic) | | | | | | | | | |
| Testosterone | Androgen | | 1 | | | 1 | | | | 2 |
| Hexobarbitol | Anesthetic | | | | 2 | 1 | | | | 3 |
| | (intravenous) | | | | | | | | | |
| Cyclexedrine | Anorexic | 1 | | | | | | | | 1 |
| Niclosamide | Antihelmintic | | | | 1 | | 1 | | 1 | 3 |
| | (cestodes) | | | | | | | | | |
| Mebendazole | Antihelmintic | 2 | | | 1 | 1 | | | | 4 |
| | (nematodes) | | | | | | | | | |
| Amphotalide | Antihelmintic | 1 | | | 1 | 1 | | | 1 | 4 |
| | (schistosoma) | | | | | | | | | |
| Retinoic acid | Antiacne | | | 1 | | | | | | 1 |
| Emetine | Antiamebic | 1 | | | | | | | 4 | 5 |
| Nifedipine | Antianginal | 1 | | | | 2 | | | 1 | 4 |
| Quinidine | Antiarrhythmic | 2 | 1 | | | | | | 1 | 4 |
| Chloramphenicol | Antibiotic | | 1 | | 1 | | | | 1 | 3 |
| | (amphenicol) | | | | | | | | | |
| Rifamide | Antibiotic | | 2 | | 2 | 3 | 2 | | 4 | 13 |
| | (ansamycin) | | | | | | | | | |
| Ampicillin | Antibiotic | 1 | | 1 | 2 | | | | 1 | 5 |
| | (lactam) | | | | | | | | | |
| Erythromycin A | Antibiotic | 1 | 5 | | | 2 | | | 4 | 12 |
| | (macrolide) | | | | | | | | | |
| Tetracyclin | Antibiotic | 1 | 4 | | 1 | 2 | 1 | | | 9 |
| | (tetracycline) | | | | | | | | | |
| Ciprofloxacin | Antibacterial | 3 | | 1 | | 1 | | | | 5 |
| | (quinolone) | | | | | | | | | |
| Sulfamoxole | Antibacterial | 2 | | | | | | | 2 | 4 |
| | (sulfonamide) | | | | | | | | | |
| Dapsone | Antibacterial | 2 | | | | | | | 1 | 3 |
| | (sulfone) | | | | | | | | | |
| Atropine | Anticholinergic | 1 | 1 | | | 1 | | | | 3 |
| Warfarin | Anticoagulant | | 1 | | | 2 | | | | 3 |
| Nitrazapem | Anticonvulsant | 1 | | | 1 | | | | 1 | 3 |
| Zometapine | Antidepressant | 4 | | | | | | | | 4 |
| Glyburide | Antidiabetic | | | | 3 | | | | 2 | 5 |
| Uzarin | Antidiarrheal | | 7 | | | 1 | | | 1 | 9 |
| Aspirin | Anti- | | | 1 | | 1 | | | | 2 |
| | inflammatory | | | | | | | | | |
| Taxol | Antineoplastic | | 3 | | 1 | 5 | | | 1 | 10 |
| Etiposide | Antineoplastic | | 2 | | | 1 | 1 | | 8 | 12 |
| Dantrolene | Skeletal muscle | 1 | | | 2 | | | | | 3 |
| | relaxant | | | | | | | | | |

It is clear from this tabulation that “low water solubility” is not synonomous with “lipid-soluble”, since simple lipid or surfactant bilayers or monolayers will not offer the proper milieu for the solubilization of many, if not most, of these complex actives at levels meaningful for pharmaceutical application. The table rather indicates the need for solvent systems designed with these sorts of “schizophrenic” compounds in mind.

The first polar organic material may comprise a sugar having at least two hydroxyl groups or an alcohol of two or three carbons having at least two hydroxyl groups or both. The first polar organic material may have a ratio of hydroxyl groups to carbon atoms of from about 1:2 to about 1:1, preferably from about 3:5 to about 1:1 and more preferably from about 2:3 to about 1:1. Examples of suitable first liquid polar organic solvents are the following (including the ratios of hydroxyl groups to carbon atoms together with the actual numbers of hydroxyl groups and carbon atoms in parentheses for the specific compounds):

Glycerol 1:1 (3:3); Ethylene glycol 1:1 (2:2); Propylene glycol 2:3; Sorbitol 1:1 (6:6); Mannitol 1:1 (6:6); Glucose 5:6; Fructose 5:6; Sucrose 2:3 (8:12); Trehalose 2:3 (8:12); Maltose 2:3 (8:12)

The second polar organic material may comprise an amide group or an ammonium group or both, preferably, said polar organic material comprising an hydroxyl-free polar organic material comprising an amide group or an ammonium group or both. Examples of suitable second polar organic materials are the following:

acetamide, N-methylacetamide (MAA), N,N-dimethylacetamide (DMAA, also sometimes abbreviated as DMAC), and ethylammonium nitrate.

Preferably, at least some of the foregoing types of low-solubility compounds having pharmacological activity can be solubilized using a pharmacologically-acceptable mixture of polar, water-miscible solvents that comprise a glycerol and an acetamide, with mixtures of a glycerol, such as 1,2,3-propanetriol (glycerol) or 1,2-propanediol (propylene glycol), and N,N-dimethylacetamide (DMAA) or N-methylacetamide (MAA) being of particular value.

The ratio of hydroxyl compound (“H”) to amide solvent (“A”), as a weight ratio, may be generally in a range from about 1:2 to about 10:1, preferably in a range from about 1:1 to about 5:1, and more preferably in a range from about 3:2 to about 4:1.

The solvent system of the present invention may further comprise a lipid material or a surfactant material or both.

For a lipid-free solution of the foregoing poorly soluble compounds where the solution is not a liquid crystalline material, the amount of DMAA in a single administered dose should be less than about 2 grams. The ratio of glycerol to DMAA, on a weight basis, should lie in the range of 1 to 10 and preferably in the range of 1.5 to 4. This determines the minimum concentration of such compound. This concentration preferably should be such that a therapeutic dose should dissolve in a glycerol-DMAA mixture containing less than about 2 grams of DMAA, so if R is the weight ratio of glycerol to DMAA, and the therapeutic dose is set at D grams, then the minimum concentration of drug in the overall drug-glycerol-DMAA component of the formulation must be at least D/(2+2R+D).

For formulations intended for parenteral administration, the amount of DMAA in a single administered dose should preferably be less than about 2 grams, which is approximately the amount of DMAA in a single injected does of the currently marketed Vumon^{R} formulation. For oral formulatioins, higher amounts are possible, but the amount should preferably be below 5 gm, and more preferably below 2 grams. The ratio of glycerol to DMAA, on a weight basis, should preferably lie in the range of 1 to 10 and more preferably in the range of 2 to 4. If the formulation is lipid- or surfactant-based, the ratio of lipid (plus hydrophobic components, if present) to DMAA plus glycerol should be between about 0.5 and 10, preferably between about 0.7 and 4. The total amount of lipid required to formulate a single therapeutic dose of the active should be less than about 10 grams, and preferably between about 0.1 and 4.

The solvent DMAA has been found be an excellent solvent for a very wide range of organic compounds, and is even miscible with alkanes. As described in this section and elsewhere, there are a number of compelling reasons why mixtures of DMAA with glycerol, or more generally mixtures of first and second polar organic materials as defined herein, can be superior to DMAA (or second polar organic material) alone in a formulation of a compound having pharmacological activity. Some of these are now described, using the case where the first polar organic material is glycerol and the second is DMAA to simplify the discussion:

A. Often the glycerol is very beneficial as a co-solvent, helping to solubilize drugs that would be poorly soluble in DMAA alone;

B. Or, if the glycerol as co-solvent merely reduces the quantity of DMAA required for the solubilization, this is extremely valuable because of the much lower toxicity (and longer history of use) of glycerol as compared to DMAA;

C. In cases where the viscosity of the formulation is important in determining the rate of administration, such as injection, glycerol can be useful as a means to control the administration rate and thus the tendency to precipitate upon administration;

D. Very significantly, a glycerol-DMAA mixture is superior to DMAA in forming lyotropil, liquid crystals and other nanostructured phases when combined with lipids and/or surfactants; and

E. In cases where the glycerol-DMAA mixture is in equilibrium with a nanostructured lipid-based or surfactant-based phase, a drug with an oil-water partition coefficient (K_{ow}) significantly greater than unity can be made to partition preferentially into the nanostructured phase; this surfactant-based system comprising a DMAA-glycerol mixture can then provide the basis for a drug-delivery system, for example involving encapsulation of the nanostructured phase, which can be of potentially high utility;

F. Since the addition of glycerol increases the volume of total solvent, typically by at least 3-fold, any precipitating effect by water (either for injection or a body fluid such as blood) will generally require longer contact with the water or body fluid, an effect which is furthermore enhanced by the viscosity increase from the glycerol and resultant slowing of the kinetics of mixing; and

G. the previous advantage is particularly pronounced in the case where a liquid crystalline system is made possible by the inclusion of glycerol, since mixing of water or blood with polar liquid confined in the nanometer-scale pore systems of reversed liquid crystalline systems (reversed cubic and reversed hexagonal phases) is extremely slow compared to mixing in bulk.

As an example of point D, a moderately unsaturated phosphatidylcholine (PC) such as Epikuron 200 (from Lucas-Meyer) will dissolve to a liquid in an equal volume of DMAA, but upon addition of three volumes of glycerol to this mixture, a liquid crystal will form that is in equilibrium with excess glycerol-DMAA mixture, at temperatures at or near room temperature for common PC sources such as soy PC. This is readily explained in terms of the solubility of phospholipid in a more amphiphilic solvent such as DMAA, whereafter the PC is, in essence, “precipitated” by the glycerol (a non-solvent for PC), albeit the “precipitation” is to a liquid crystalline phase, rather than to a crystalline phase.

With little or no modification, these same advantages hold rather broadly for mixtures of other hydroxyl-rich solvents (first polar organic material) with amide solvents (second polar organic material) in accord with this invention. The importance of the effect of the hydroxyl-rich compound in modulating the interactions of amide solvents with lipids and surfactants is difficult to fully appreciate unless one has witnessed the drastic effect of amide solvents, such as dimethylacetamide, in undiluted form, on lipid and surfactant systems that would otherwise form well-ordered phases, such as nanostructured liquid crystalline phases. The fact that diluting an amide solvent such as DMAA with a hydroxyl-rich solvent such as glycerol (for example, at ratios of roughly 3:1 glycerol:DMAA) can prevent the liquefaction of these liquid crystals, and yet do so without precipitating the pharmaceutical active, is a very surprising result and one of the main thrusts of this invention.

In terms of solubilization of actives (with or without the presence of surfactants), there can be considerable synergy in combining the hydroxyl-rich compounds and amide compounds as per this invention. The hydroxyl-rich compounds can act as hydrogen-bonding donors with the active to be dissolved, while the amide can serve as donor or acceptor, meaning that both hydrogen-bonding acceptor and donor groups in the active can readily find groups in the solvent mixture to hydrogen bond with; favorable solvent-solute interactions of course favor dissolution. From a toxicity point of view, amides as a class tend to be less readily accepted when used in large amounts (particularly for injectables, but even in oral formulations), whereas the extremely low toxicities of compounds like glycerol and sugars permit their use in much larger quantities; thus the use of a minimal amount of amide, complemented with a liberal amount of glycerol or sugar or other hydroxyl compound, is advantageous from a toxicity viewpoint. And in addition, both amide groups and hydroxyl groups are only moderately polar, as evidenced by the fact that they are listed as polar groups “not operative as surfactant head groups” in the important review of surfactant head group requirements by Laughlin (see R. Laughlin, Advances in liquid crystals, vol. 3, p. 41, 1978). Broadly speaking, one would not want to invoke a solvent with a strongly polar group, one operative as a surfactant head group, in the solubilization of an active with low water solubility. In short, the combination of amide with sugar provides a milieu rich in hydroxyl groups, carbonyls, and amides with their N—H bonds but without (high concentrations of) strongly polar groups that might “scare off” what are fundamentally hydrophobic actives.

For the cases where the hydroxyl-rich compound is a sugar, it is also worth noting that sugars in general, and certain sugars such as trehalose in particular, have a protective effect when used in lipid-containing formulations. For example, trehalose has been shown to prevent bilayer rearrangements that can occur as the result of temperature excursions below the freezing point of water, for example.

Molecular weight plays an important role in determining the effectiveness of these solvent combinations, with the general rule being that lower MW solvents are more effective than higher MW. This is in part due to larger, more favorable entropy of mixing that follows from the higher molar quantities per unit volume, at lower MW. Broadly speaking, the MW of most good solvents is below 500 D, preferably below 250, and in fact most preferably below about 150 D. Thus, although solvents such as polyethyleneglycol (PEG) could be useful in the practice of this invention in some circumstances, low-MW solvents such as glycerol (MW=92) are preferred.

The mixtures of polar solvents disclosed herein are useful for solubilizing not only dantrolene and phenytoin but more generally at least some of the chemically-related compounds having pharmacological activity, containing one or more of the chemical groups that distinguish dantrolene not only pharmacologically but also in terms of polar intermolecular interactions that are important in determining crystal energies and solubility properties: namely a nitrophenyl group (or the closely related nitrofuranyl group), a hydantoin group (or analogously a substructure containing one amide and a nitrogen or sulfur atom separated by a single carbon, or at least two nitrogen atoms separated by a single carbon), or the pi orbital-rich group —C═N—N—C═O—. These include the following classes of compounds:

1. Containing a hydantoin-group:

a. Dantrolene (skeletal muscle relaxant)(also has nitrophenyl group)

b. Phenytoin (anticonvulsant)

c. Fosphenytoin (anticonvulsant)

2. Containing an amide group and one polar atom, N or S, separated by a single carbon and a nitrophenyl- or nitrofuranyl-group:

a. Nimetazepam (skeletal muscle relaxant)

b. Nitrazepam (skeletal muscle relaxant)

3. Containing one amide group and one polar atom, N or S, separated by a single carbon:

a. Afloqualone (skeletal muscle relaxant)

b. Chlormezanone (skeletal muscle relaxant)

c. Diazepam (skeletal muscle relaxant)

d. Flumetramide (skeletal muscle relaxant)

e. Tetrazepam (skeletal muscle relaxant)

4. Containing two nitrogen atoms separated from each other by a single carbon:

a. Phenyramidol (skeletal muscle relaxant)

b. Tizanidine (skeletal muscle relaxant)

c. Zoxazolamine (skeletal muscle relaxant)

5. Containing an amide group as part of a 5-member ring:

a. Mephenoxalone (skeletal muscle relaxant)

b. Metaxalone (skeletal muscle relaxant)

6. Containing both a hydantoin- and a nitrophenyl- or nitrofuranyl-group:

a. Nifurtoinol (antibiotic)

b. Nitrofurantoin (antibiotic)

7. Containing a nitrophenyl- or nitrofuranyl-group and a —C═N—N—C═O— group:

a. Furaltadone (antibiotic)

b. Furazolidone (antibiotic)

c. Nifuradene (antibiotic)

d. Nifuratel (antibiotic)

e. Nifurfoline (antibiotic)

8. Containing a nitrophenyl- or nitrofuranyl-group:

a. Nimodipine (antibiotic)

b. Nifurpirinol (antibiotic)

c. Nifurprazine (antibiotic)

d. Nitrosulfathiazole (antibiotic)

9. Containing a terminal nitrofuranyl-group, and two nitrogens separated from each other by a single carbon:

a. Furazolium chloride (antibiotic)

10. Containing a plurality of nitrophenyl- or nitrofuranyl-groups

a. Nimopidine (cerebral vasodilator)

b. Nitracrine (antineoplastic)

c. Nitrefazole (alcohol deterrent)

d. Nitrendipine (antihypertensive)

e. Nitrodan (anthelmintic)

The following examples illustrate the present invention but are not to be construed as limiting the invention.

The present application also encompasses the administration of poorly soluble compounds to a patient. The poorly soluble compound is solubilized in a solvent system as described herein, and may be administered by any of the many means that are well-known to those of skill in the art. Such means include but are not limited to orally (for example, in the form of a liquid, pill, capsule, lozenge, etc.), parenterally (e.g. via injection, intravenously, etc.), transdermally, intraocularly, rectally via suppository, and buccal. In preferred embodiments of the invention, the poorly soluble compound is dantrolene or salts thereof, or dilantin.

### EXAMPLES

### Example 1

Sodium 1-[[[5-(4-Nitrophenyl)-2-furanyl]-methylene]amino]-2,4-imidazolidinedione hemiheptahydrate (sodium dantrolene) is poorly soluble in water. A single 300 mg dose requires approximately 1 liter of water for complete dissolution. In contrast, 0.0053 gm of sodium dantrolene was found to be soluble in a mixture of 0.0219 gm DMAA and 0.0543 gm glycerol. The glycerol greatly facilitated the dissolution of the sodium dantrolene, which took only seconds and required no heating. This means that the same 300 mg dose of sodium dantrolene can be dissolved in 1.24 gm of DMAA plus 3.07 gm of glycerol. Previously a 300 mg dose of sodium dantrolene was required to be reconstituted in approximately 15 vials using 60 ml water for each vial. During treatment of critical malignant hyperthermia by injection of aqueous sodium dantrolene, a circumstance in which response time is crucial to survival of the patient. The time saved by reducing this to a single injection of less than 10 ml. is significant, and could result in saving lives that would otherwise be lost in the hast to prepare large numbers of high-volume injections.

The highest loading of dantrolene achievable in a system of the present invention is approximately 25% by weight; indeed, for example, in the present Example the 0.0053 gm of sodium dantrolene completely dissolved in the 0.0219 gm of DMAA. On the other end of the spectrum, the concentration of the DMAA-glycerol solvent system in an overall formulation could be as low as about 1%, even when the total amount of formulation is held to under about 100 ml (that is, on the order of 1 gram of DMAA-glycerol solvent system would be used to dissolve the therapeutic dose of about 200 mg, and the remainder of the 100 ml could be, for example, water in which microcapsules containing the solvent system and the dantrolene were dispersed).

### Example 2

A liquid crystalline material was first prepared as follows: Imidazole, in the amount of 0.0853 gm, was added to 0.5592 gm of a 3:1 glycerol:DMAA (by wt) mixture, followed by 0.3516 gm of egg phosphatidylcholine (egg PC), 0.0635 gm of essential oil of sandalwood, and 0.0076 gm of octadecylamine. Each of these compounds is of low toxicity even via intravenous route. After mixing and equilibrating this mixture, 0.2248 gm of the mixture was combined with 0.0044 gm of sodium dantrolene. On equilibration, nearly all of the sodium dantrolene was solubilized in the lipid-rich phase, which was a lyotropic liquid crystalline phase at 4 degrees Centigrade. This experiment thus demonstrates the utility of a glycerol:DMAA mixture in providing for a dantrolene-solubilizing liquid crystalline matrix when combined with phospholipid and other secondary excipients. The need for octadecylamine is reduced by the use of phospholipid mixtures that contain a lower concentration of acidic lipids than the egg PC used in this experiment.

### Example 3

A dantrolene-containing liquid crystalline material of similar composition to that of Experiment 2 was mixed gently with 3 parts by weight of a 3:1 glycerol:DMAA mixture, after which the test tube containing the entire mixture was allowed to equilibrate for 48 hours. Centrifugation was then used to separate the liquid crystalline phase from the excess glycerol:DMAA mixture. It was found that the liquid crystalline phase contained the vast majority of the dantrolene sodium, with much smaller amounts present in the glycerol:DMAA-rich phase, meaning that the dantrolene sodium preferentially partitioned into the lipid-rich phase.

### Example 4

The hydantoin-containing anticonvulsant drug phenytoin (trade name Dilantin), chemically diphenylhydantoin, in the amount 0.1023 gm, was dissolved in a mixture of 0.5733 DMAA and 1.2490 gm glycerol. The ratio of glycerol to DMAA was thus 2.17:1. The lower glycerol content in this phenytoin-solubilizing mixture as compared to that in the case of the dantrolene-solubilizing mixture described in Experiment 1 is readily explained on the basis of the greater hydrophobicity of phenytoin. The concentration of phenytoin in this solution was 5.3 wt %.

For phenytoin, the glycerol:DMAA ratio may be less than or equal to about 2.2:1 in order to solubilize concentrations of phenytoin of about 5% or greater. Dantrolene, however, is able to tolerate higher glycerol:DMAA ratioes while maintaining solubilities of at least several weight percent, but a practical limit of about 5:1 is operative.

### Example 5

This experiment simulates the conditions present when an aqueous solution of dantrolene sodium in glycerol:DMAA is added to physiologic fluids, such as human blood plasma, for administration, such as by intravenous injection, so as to test whether or not precipitation of dantrolene in the physiologic fluid would be likely to occur. Dantrolene sodium, in the amount 0.0212 gm, was dissolved in a mixture of 0.2177 gm glycerol and 0.0909 gm DMAA. One hundred milliliters of citrate-buffered human blood plasma was circulated through ¼″ silicone tubing with a peristaltic pump, and 0.0913 gm of the dantrolene-containing mixture (thus containing 6 mg dantrolene sodium) was injected into the circuit. At a distance of 25″ from the site of injection, a 0.5 micron in-line filter was present to capture any precipitated dantrolene crystals. The flow rate was adjusted to approximately 50 ml/minute. Approximately 15 seconds after the time of injection, the flow was stopped, the filter removed and purged with air, and the filter was then examined for dantrolene crystals in a polarizing optical microscope. No crystals nor orange color were detected on the filter, neither by eye, nor with a 14× magnifying piece, nor in the microscope at 40×, 100×, or 400×. The remaining plasma in the 25″ length of tubing was strongly orange from the dantrolene, but no crystals were evident there as well.

### Example 6

High fructose corn syrup, in the amount of 1.525 gm, was mixed with 0.50 gm N,N dimethylacetamide to form a liquid. Into 0.065 gm of this mixture was dissolved 0.0037 gram of dantrolene sodium. This constitutes a 5.7% solution of dantrolene sodium, meaning that an active dose of 200 mg could be dissolved in less than 4 ml of total injectable solution. Furthermore, the sugars present in the solution may play a role in ameliorating renal problems associated with the malignant hyperthermia condition for which dantrolene is the indicated drug.

It is apparent that many modifications and variations of the invention may be made without departing from the spirit and scope of the present invention. It is understood that the invention is not confined to the particular construction and arrangement herein described, but embraces such modified forms of it as come within the appended claims. The specific embodiments described are given by way of example only and the invention is limited only by the terms of the appended claims.

I claim:

1. A composition comprising
a poorly soluble compound having pharmacological activity, said poorly soluble compound requiring more than about 100 ml of water to effect solubilization of a therapeutic amount of the compound in the absence of a solvent system; and
a solvent system comprising
a. a first polar organic material comprising a sugar having at least two hydroxyl groups or an alcohol of two or three carbons having at least two hydroxyl groups, or both, and
b. a second polar organic material comprising an amide group or an ammonium group or both.

2. The composition of claim 1 further comprising a lipid material or a surfactant material or both.

3. The composition of claim 1, wherein said first polar organic material has a ratio of hydroxyl groups to carbon atoms of from about 1:2 to about 1:1.

4. The composition of claim 1, wherein said first polar organic material has a ratio of hydroxyl groups to carbon atoms of from about 3:5 to about 1:1.

5. The composition of claim 1, wherein said first polar organic material has a ratio of hydroxyl groups to carbon atoms of from about 2:3 to about 1:1.

6. The composition of claim 1, wherein said first polar organic material comprises a sugar.

7. The composition of claim 1, wherein said first polar organic material comprises a dihydric or trihydric alcohol of two or three carbons.

8. The composition of claim 7, wherein said alcohol comprises a glycerol or a glycol.

9. The composition of claim 1, wherein said second polar organic material comprises an hydroxyl-free polar organic material comprising an amide group or an ammonium group or both.

10. The composition of claim 9, wherein said second polar organic material comprises an amide.

11. The composition of claim 10, wherein said amide comprises an acetamide.

12. The composition of claim 1, wherein said second,polar organic material comprises N,N-dimethylacetamide.

13. The composition of claim 1, wherein said poorly soluble compound has one or more polar groups in its molecular structure, said polar groups comprising
a. a hydantoin-group;
b. an amide group and one polar atom, N or S, separated by a single carbon and a nitrophenyl- or nitrofuranyl-group,
c. an amide group and one polar atom, N or S, separated by a single carbon;
d. two nitrogen atoms separated from each other by a single carbon;
e. an amide group as part of a 5-member ring;
f. both a hydantoin- and a nitrophenyl- or nitrofuranyl-group;
g. a nitrophenyl- or nitrofuranyl-group and a —C═N—N—C═O— group;
h. nitrophenyl- or nitrofuranyl-group;
i. a terminal nitrofuranyl-group, and two nitrogens separated from each other by a single carbon and;
j. a plurality of nitrophenyl- or nitrofuranyl-groups.

14. The composition of claim 1, wherein the ratio of the first polar organic material to the second liquid polar organic material, as a weight ratio, is in a range from about 1:2 to about 10:1.

15. The composition of claim 14, wherein said ratio is in a range from about 1:1 to about 5:1.

16. The composition of claim 14, wherein said ratio is in a range from about 3:2 to about 4:1.

17. The composition of claim 1, in which the first polar organic material is a liquid.

18. The composition of claim 1, in which the second polar organic material is a liquid.

19. The composition of claim 1, in which both the first polar organic material and the second polar organic material are liquids.

20. The composition of claim 1 wherein said solvent system is non-aqueous.

21. The composition of claim I wherein said first polar organic material has a molecular weight of less than about 150.

22. A solvent system for the solubilization of a poorly soluble compound having pharmacological activity, said system comprising
a. a dihydric or trihydric alcohol of two or three carbons and
b. one or more of acetamide, ethylammonium nitrate, N-methylacetamide and dimethylacetamide.

23. A method for solubilizing a poorly soluble compound comprising the steps of
combining a poorly soluble compound having pharmacological activity, said poorly soluble compound requiring more than about 100 ml of water to effect solubilization of a therapeutic amount of the compound in the absence of a solvent system, with a solvent system comprising
a. a first polar organic material comprising a sugar having at least two hydroxyl groups or an alcohol of two or three carbons having at least two hydroxyl groups or both and
b. a second polar organic material comprising an amide group or an ammonium group or both,
said solvent system being present in an amount sufficient to solubilize said poorly soluble compound; and
dissolving said poorly soluble compound with said solvent system.

24. A method of providing to a patient in need thereof a poorly soluble compound having pharmacological activity, comprising the step of
administering to said patient a pharmaceutical composition comprising
i. said poorly soluble compound, and
ii. a solvent system comprising
a. a first polar organic material comprising a sugar having at least two hydroxyl groups or an alcohol of two or three carbons having at least two hydroxyl groups or both, and
b. a second polar organic material comprising an amide group or an ammonium group or both.

25. The method of claim 24 wherein said solvent system further comprises a surfactant, and said step of administering is oral.

26. A solvent system for the solution of compounds containing at least one hydantoin group in a polar solvent,
said solvent system comprising
a. a dihydric or trihydric alcohol of two or three carbons and
b. a polar organic material comprising an amide group or an ammonium group or both.

27. The solvent system of claim 26, wherein said compound containing at least one hydantoin group is dantrolene.

28. The solvent system of claim 26, wherein said compound containing at least one hydantoin group is dilantin.

29. A pharmaceutical composition, comprising:
a compound containing at least one hydantoin group, and
a solvent system comprising
a. a first polar organic material comprising a sugar having at least two hydroxyl groups or an alcohol of two or three carbons having at least two hydroxyl groups or both, and
b. a second polar organic material comprising an amide group or an ammonium group or both,
said dantrolene being dissolved in said solvent system.

30. The solvent system of claim 29, wherein said compound containing at least one hydantoin group is dantrolene.

31. The solvent system of claim 29, wherein said compound containing at least one hydantoin group is dilantin.

32. A solvent system for the solution of dantrolene and salts thereof in a polar solvent, said solvent system comprising
a. a dihydric or trihydric alcohol of two or three carbons and
b. a polar organic material comprising an amide group or an ammonium group or both.

33. A solvent system in accordance with claim 32, wherein said solvent system comprises
a. 1,2,3-propanetriol and
b. N,N-dimethylacetamide.

34. A pharmaceutical composition, comprising:
dantrolene or salts thereof, and
a solvent system comprising
a. a first polar organic material comprising a sugar having at least two hydroxyl groups or an alcohol of two or three carbons having at least two hydroxyl groups or both, and
b. a second polar organic material comprising an amide group or an ammonium group or both,
said dantrolene being dissolved in said solvent system.

35. The pharmaceutical composition of claim 34, wherein said dantrolene is present in a range of from about 0.3% to about 25%.

36. The pharmaceutical composition of claim 34, wherein said solvent system is present in a range of from about 1 to about 99%.

37. The pharmaceutical composition of claim 34, wherein said first polar organic material is selected from the group consisting of glycerol, a sugar and a plurality of sugars.

38. The pharmaceutical composition of claim 34, wherein said second polar organic material is selected from the group consisting of acetamide, N-methylacetamide, and N, N-dimethylacetamide.

39. A method of providing dantrolene to a patient in need thereof, comprising the step of administering to said patient a pharmaceutical composition comprising
i. dantrolene or salts thereof, and
ii. a solvent system comprising
a. a first polar organic material comprising a sugar having at least two hydroxyl groups or an alcohol of two or three carbons having at least two hydroxyl groups or both, and
b. a second polar organic material comprising an amide group or an ammonium group or both,
said dantrolene being dissolved in said solvent system.

40. A solvent system for the solution of dilantin in a polar solvent, said solvent system comprising
a. a dihydric or trihydric alcohol of two or three carbons and
b. a polar organic material comprising an amide group or an ammonium group or both.

41. A solvent system in accordance with claim 40, wherein said solvent system comprises
a. 1,2,3-propanetriol and
b. N,N-dimethylacetamide.

42. A composition for the solubilization of poorly-soluble compounds comprising a nanostructured liquid or nanostructured liquid crystalline phase, comprising:
a solvent system comprising
a. a first polar organic material comprising a sugar having at least two hydroxyl groups or an alcohol of two or three carbons having at least two hydroxyl groups, or both, and
b. a second polar organic material comprising an amide group or an ammonium group, or both, and
a lipid or surfactant.
